(19) United States
(12) Patent Application Publication
(10) Pub. No.: US 2009/0143632 A1
(43) Pub. Date: Jun. 4, 2009

(54) SORBENTS AND PROCESSES FOR SEPARATION OF OLEFINS FROM PARAFFINS

(76) Inventor: Marianna F. Asaro, Belmont, CA (US)

Correspondence Address: Wall & Tong, LLP;SRI INTERNATIONAL, 595 SHREWSBURY AVENUE, SHREWSBURY, NJ, 07702, US

(21) Appl. No.: 12/326,734

(22) Filed: Dec. 2, 2008

Related U.S. Application Data
(60) Provisional application No. 60/992,168, filed on Dec. 4, 2007.

(51) Int. Cl. C07F 15/00; C07C 7/12

(52) U.S. Cl. 585/823; 546/12; 556/136; 585/850

(57) ABSTRACT

In one embodiment, the present invention relates generally to a method for separating olefins from paraffins. In one embodiment, the method includes providing a mixture comprising olefins and paraffins, providing a gas separation agent to associatively, reversibly and selectively bind the olefin and dissociating the olefin from the gas separation agent.

### RELATED APPLICATIONS

This application claims priority under 35 U.S.C. § 119(e) to U.S. provisional patent application Ser. No. 60/992,168, filed on Dec. 4, 2007, which is hereby incorporated by reference in its entirety.

### REFERENCE TO GOVERNMENT FUNDING

This invention was made with Government support under contract number DE-FC36-04GO14151 awarded by the Department of Energy. The Government has certain rights in this invention.

### FIELD OF THE INVENTION

The present invention relates generally to carriers and processes for separation of olefins from paraffins.

### BACKGROUND OF THE INVENTION

Separation of olefins (e.g. ethylene, propylene, etc.) from paraffins (e.g. ethane, propane, etc.) is among the most expensive industrial separations. Generally, the separation of olefins from paraffins is performed in a distillation column. Due to the very close boiling points of the olefin and paraffin, these distillation columns are the largest known in commercial use. The energy demand of these separations is estimated at 120 trillion British Thermal Units (BTU)/year and is considered extremely cost intensive.

A gas separation technology alternative to distillation in principle would be use of molecular sieves in a pressure swing adsorption (PSA) device. However, this is not considered seriously for separation of olefins from paraffins because the sizes and polarities of the olefin and paraffin are so close that sieving gives either 1) very low purity at high recovery, or 2) very low recovery at high purity, neither situation being economically attractive for commercial olefin/paraffin separation at any scale.

Certain polymeric membrane materials show some selectivity for one component over the other, but these are prevented from commercialization due to various issues. One issue is that the selectivity of the polymers is not high enough. The selectivity of membrane materials is based on a combination of solubility, which is similar for olefins and their corresponding paraffins, and diffusivity, which again suffers the limitation to selectivity caused by the very similar size and polarity of the olefin and corresponding paraffin. A second issue is that the effective membrane thickness that allows for good flux is too small to allow for good throughput. A further reason is that polymeric membranes tend to be mechanically weak. The selectivity, recovery and capacity issues require improvements to both membrane materials and separator designs.

### SUMMARY OF THE INVENTION

In one embodiment, the present invention is directed toward a gas separation agent for separating an olefin from a paraffin by reversibly and selectively binding said olefin. In one embodiment, the gas separation agent has a formula comprising:

[M(Xₐ)(X_{b})(Y-L-Y′)]ⁿz[counterion]^{m},

wherein M is a metal having a vacant coordination site for associatively binding said olefin to separate said olefin from said paraffin, wherein Xₐ is at least one of: a mono-anionic ligand or a donor ligand, wherein X_{b} is at least one of: a mono-anionic ligand or a donor ligand, wherein Y-L-Y′ is a bidentate non-anionic ligand having two moieties Y and Y′, wherein counterion is an ion for balancing a charge of the gas separation agent, wherein a and b are integers greater than or equal to zero and wherein n, m and z have a charge balance relationship of n+zm=0.

In one embodiment, the present invention is directed toward a second embodiment of a gas separation agent for separating an olefin from a paraffin by reversibly and selectively binding said olefin. In the second embodiment, the gas separation agent has a formula comprising:

[ML₁L₂L₃L₄]ⁿz[anion]^{m},

wherein M is a metal having a vacant coordination site for associatively binding said olefin to separate said olefin from said paraffin and wherein each one of L₁-L₄ are a datively bonded, covalent donor ligand, wherein anion is an ion for balancing a charge of the gas separation agent and wherein n, m and z have a charge balance relationship of n+zm=0.

In one embodiment, the present invention is directed toward a method for separating olefins from paraffins. In one embodiment, the method comprises providing a mixture comprising olefins and paraffins, providing a gas separation agent to associatively, reversibly and selectively bind said olefin and dissociating said olefin from said gas separation agent.

### BRIEF DESCRIPTION OF THE DRAWINGS

So that the manner in which the above recited features of the present invention can be understood in detail, a more particular description of the invention may be had by reference to embodiments, some of which are illustrated in the appended drawing. It is to be noted, however, that the appended drawing illustrates only typical embodiments of this invention and are therefore not to be considered limiting of its scope, for the invention may admit to other equally effective embodiments.

FIG. 1 depicts a flow diagram of one embodiment of a method for separating olefins from paraffins.

### DETAILED DESCRIPTION

The present invention provides carrier compounds or gas separation agents for separation of olefins from paraffins. The gas separation agents (hereinafter also referred to as sorbents, e.g., either adsorbents or absorbents) may be organometallic or inorganic compounds. The novel gas separation agents disclosed herein provide solutions to the problems noted above in the background. One additional advantage to the novel gas separation agents described herein is that their use achieves separation of olefins from paraffins without a threat of explosion found for previously used silver and copper sorbents. The olefins are unsaturated hydrocarbons, also referred to herein as alkenes. The paraffins are saturated hydrocarbons, also referred to herein as alkanes.

Currently used complexes may contain silver that catalyzes formation of acetylide polymers from acetylene present as an impurity in commercial olefin and paraffin feeds. Silver is known to rapidly polymerize acetylene with explosive violence.

For example, the novel gas separation agents described herein do not catalyze formation of acetylene polymers during separation of olefins from paraffins. Acetylene polymers are not formed in the presence of acetylene impurity found in commercial olefin and paraffin feeds when these gas separation agents are used. As a result, the potential threat of explosion with silver or copper is eliminated by the use of the novel gas separation agents of the present invention.

Another advantage of the novel gas separation agents described herein is that their gas separation properties may be tuned. In one embodiment, the gas separation agents may be tuned by altering structures and compositions of the gas separation agent. For example, equilibrium constants and kinetic parameters may be optimized. Currently used gas separation agents that include silver possess binding constants for olefin that may be too high for commercial utility. By altering the structures and compositions of the novel complex, key performance parameters such as the binding constant and rate constant can be rationally tuned to meet specifications of a process feedstream and process requirements of purity, recovery, and cost. Target values or ranges of binding constants, mass transfer coefficients, and heat transfer coefficients may be gleaned through use of a computer program that relates and iterates these sorbent properties with the design features of a separator and the process requirements. The computer program may be a first level correlation, using an algebraic program written in, for example Excel®, or the computer program may provide a more detailed correlation obtained by iterative convergence using a problem solver program, such as for example, MultiPhysics. The structure and composition of the tunable sorbent is selected based on these iterations and the tunability of the novel sorbents described herein.

In one embodiment, the tuning of the gas separation agent is based on theory that a molecular and electronic structure of an olefin gas separation agent (e.g. an olefin bonded to a gas separation agent described herein) strongly influences properties of the gas separation agent, according to ligand field theory and the Chatt-Dewar-Duncanson model of olefin binding. Rational design to affect binding properties is based on Structure Activity Relationships (SARs), wherein trends are observed in performance, or activity, of the gas separation agent in parallel with the introduction of systematic changes to the molecular and/or electronic structure of the gas separation agent. Therefore, it stands to reason that the structure of a gas separation agent can be modified by rational design to affect its olefin binding properties and, thereby, its performance as an olefin/paraffin separation material.

SARs may arise from changes to the electron density at an olefin binding site, from steric (spatial) effects around the olefin binding sites, or from combinations thereof. Experimentally determined SARs may arise from either or both of these effects. The theoretical origin of a SAR is assessed based on correlation with known principles of olefin binding. The combination of SAR data and knowledge of the state of the art in ligand binding theory allows construction of a rational approach to influencing properties.

Various properties of an olefin gas separation agent material may be influenced by SARs. These include binding constants (affinity for olefin), kinetic constants (rates of olefin binding and release), and solubility. While a structural change may affect multiple properties of the gas separation agent, a SAR is typically targeted to influence a single property. For complexation of olefins (i.e. binding of olefins to the gas separation agent), a primary initial screen is the equilibrium constant or, for solid sorbents, the sorption coefficient. These are both denoted herein as K. For olefin binding K=K_{bind}, and for release of olefin, K=K_{disso}, where K_{disso}=1/K_{bind}. The optimal value for K_{bind} depends on specifics of the separator type and design; kinetic factors; and the composition of the gas feed containing olefin as well as target purity and recovery for the process streams.

In one embodiment, the optimal value of K_{bind} for the invention may be between 1 and 5000 M⁻¹, when concentrations are defined in terms of molarity, as in equation (1) below. In another embodiment, the value of K_{bind} may be between 5 and 500 M⁻¹ for most applications. Preferably, the fraction of gas separation agent that is bound to olefin during sorption will be at least 10%. For K_{bind} expressed in units of pressure, values of K_{bind} may be in the range of 0.1 to 100 atm⁻¹.

$${K}_{bind} = \frac{[ olefin \text{-} gas separation agent ]}{[ olefin ] [ unbound gas separation agent ]} \qquad ( 1 )$$

The theoretical basis of the resultant SARs may be electronic, steric, or a combination thereof and can be observed as a rational change in structure causing a predictable change in a property such as the binding constant. Typically the change in binding constant is predictable in direction (increase or decrease) and range of magnitude. This tuning may be referred to as qualitative or semi-quantitative, depending on the extent to which the magnitude of changes can be predicted. Because design of an effective combination of sorbent and separator design iterates based on initial laboratory demonstration, being able to tune the value of K qualitatively or semi-qualitatively is enabling to the progressive design. Thus, the present invention provides classes of gas separation agents for which olefin binding SARs would be discernable based on theory and precedent.

Tunability of the novel gas separation agents is based on several features of the gas separation agents: (1) a metal of the gas separation agent has a partially vacant coordination sphere when the gas separation agent is in an unbound form, the vacant coordination site(s) being provided by a square planar or tetrahedral geometry that allows for associative bonding of olefin rather than a purely dissociative pathway for olefin binding. Specifically, the square planar or tetrahedral sorbent geometries (unbound form) of the invented gas separation sorbents do not require a priori that a ligand(s) bound to the sorbent dissociate prior to binding of the olefin to the sorbent; (2) trends in organometallic and inorganic chemistry that allow rational design of improved gas separation agents based on steric and electronic factors; and (3) a correlation and optimization of target values or ranges of binding constants, mass transfer coefficients, and heat transfer coefficients for sorbent/separators combinations with the process specifications of the process feedstream and the process requirements of purity, recovery, and cost.

The square planar or tetrahedral geometries of the unbound sorbent provide advantages of (1) tunability, as described above, (2) stability in the absence of olefin, and (3) stability in the absence of a coordinating ligand such as a donor solvent molecule or a donor element within a solid matrix. Such sorbent-donor interactions, present for example in aqueous silver systems or in solvated metallocubane systems, may interfere with the gas separation process by preventing or competing with sorbent-olefin interactions. Upon binding of olefin, the gas separation agents become trigonal bipyramidal or square planar in geometry, which stabilizes the structure both electronically and sterically. The specifics of molecular structure and composition allow tuning of olefin binding properties.

In one embodiment, the novel gas separation agent comprises a gas separation agent having a formula comprising:

[M(Xₐ)(X_{b})(Y-L-Y′)]ⁿz[counterion]^{m} (2)

In the embodiment illustrated by formula (2), M is a metal having a vacant coordination site when the gas separation agent is unbound. The vacant coordination site may be used for associatively binding an olefin to separate the olefin from a paraffin. In other words, the metal M does not require that a metal-ligand bond be dissociated before binding an olefin. Xₐ is at least one of a mono-anionic ligand or a donor ligand, X_{b} is at least one of a mono-anionic ligand or a donor ligand and Y-L-Y′ is a bidentate non-anionic ligand having two moieties Y and Y′. The counterion may be any ion for balancing a charge of the gas separation agent. In formula (2), a and b are integers greater than or equal to zero and n, m and z have a charge balance relationship of n+zm=0.

The novel gas separation agent of formula (2) above selectively and reversibly binds an olefin. The novel gas separation agent of formula (2) above selectively binds an olefin because the novel gas separation agent tends to only bind with an olefin from an olefin/paraffin feed stream. The novel gas separation agent of formula (2) above reversibly binds an olefin because a reaction of the olefin and the novel gas separation agent may go in either direction.

For the embodiment illustrated in formula (2), M may be a metal selected from a group consisting of cobalt (Co), rhodium (Rh), iridium (Ir), palladium (Pd), platinum (Pt) and nickel (Ni). The gas separation agent has an electronic configuration of less than 18 electrons around the metal M when unbound to the olefin.

The ligand Xₐ may be present with X_{b} also present or Xₐ may be present with X_{b} absent. The ligands Xₐ and X_{b} may be mono-anionic ligands or donor ligands or a mixture thereof. The ligands Xₐ and X_{b} may be the same or different from one another.

Monoanionic examples of Xₐ and X_{b} may include the halides, which are chlorine (Cl), bromine (Br), iodine (I) or fluorine (F). In one embodiment, one of the ligands may be a halide and the other ligand may be an alkyl group such as methyl, ethyl, propyl, isopropyl, butyl, isobutyl, tert-butyl or higher linear or branched alkyls such as hexyl or octyl. Alternatively, one ligand may be a halide and the other ligand may be an aromatic moiety such as, for example; phenyl, alkylaryl, halogenated aryl, nitrated aryl, sulfonated aryl, aminated aryl, alkoxylated aryl, ether-substituted aryl, methylidenearyl, carboxylated aryl, oxime-substituted aryl; wherein the aryl may be substituted with from 0 to 5 groups. In another embodiment, the aryl may be substituted by 0 to 2 groups. The ligands Xₐ and X_{b} may also be chalcogenide ligands alkoxides or sulfides including, but not limited to methoxy, ethoxy, phenoxy, methylidenephenoxy, thiol, methylthiol or hydroxyl.

Donor ligand examples of Xₐ and X_{b} may include solvent molecules or other small molecules including but not limited to acetonitrile, dimethylsulfoxide, pyridine, dimethylformamide, methyl amine, ethyl amine, propyl amine, butyl amine, tert-butylamine, methylpyrrolidinone or water.

As discussed above, in the embodiment of the present invention as illustrated by formula (2), the ligand Y-L-Y′ may be a bidentate non-anionic ligand having two moieties or donor ligands Y and Y′. For example, the ligand Y-L-Y′ may be considered bidentate because the ligand typically bonds at two different sites. For example, each moiety Y and Y′ are capable of forming a dative (i.e. non-ionic) donor bond to the metal M. Y and Y′ may be different moieties or may be the same.

In one embodiment, the bidentate ligand Y-L-Y′ may be a 1,2-disubstituted organic ligand, alternatively known as a vicinally substituted organic ligand, wherein the two substituents are each nicogenides (e.g., groups containing nitrogen (N), phosphorous (P), arsenic (As) or antimony (Sb)), are each chalcogenides (e.g., groups containing oxygen (O), sulfur (S) or selenium (Se)) or are one each of a nicogenide and a chalcogenide.

In another embodiment, the bidentate ligand Y-L-Y′ may also be a 1,3-substituted organic ligand having the nicogenide or chalcogenide substituents in the 1,3-position. It is also possible that the substitution pattern of section L of the ligand is 1,4-, or even higher, with these examples least common due to the enlarged bite angle at the metal M for the higher substitution patterns.

As noted above, the moieties Y and Y′ may be the same or different from one another. Y and Y′ may be amino groups contained in a diamine or diimine ligand including but not limited to ethylene diamine, propylene diamine, N,N′-tetramethylethlylene diamine, N,N′-bis(dimethylamido)-1,2-dimethylethylenediimine, 1,1,2,2-tetramethylethylenediamine, 1,2-diethyl,1,2-dimethylethylenediamine, 1,1,2,2-tetraethylethylenediamine, N,N′-dimethylethylenediamine, 1,2-phenylethylenediamine, 1,2-dimethyl,1,2-dipheylethylenediamine, bipyridyl, substituted bipyridyl, phenanthroline, substituted phenanthrolines such as 1,10-di-Gp-phenanthroline, where Gp is an organic group containing from 1 to 10 carbon atoms, 0 to 4 nitrogen atoms, 0 to 4 oxygen atoms, 0 to 4 sulfur atoms or 0 to 4 phosphorous atoms.

Alternatively, Y and Y′ may be nicogenide or chalcogenide atoms in an ortho-substitution pattern on an aromatic ring, including but not limited to where Y-L-Y′ is a catechol or a catechol having substituent(s) on the aromatic ring, orth-dimethoxybenzene or an ortho-dimethoxybenzene having substituent(s) on the aromatic ring, a 1,2-diamineobenzene or a 1,2-diamonbenzene having substituent(s) on the aromatic ring, a 1,2-bis(arseno)benzene or a 1,2-bis(arseno)benzene having substituent(s) on the aromatic ring, a 1,2-diphosphinobenzene or a 1,2-diphosphinobenzene having substituent(s) on the aromatic ring or a 1,2-bis(phosphino)benzene or a 1,2-bis(phosphino)benzene having substituent(s) on the aromatic ring.

Alternatively, Y and Y′ may be phosphorous groups contained in a substituted ethane ligand including but not limited to 1,2-(Ar′)(Ar)ethane, where Ar and Ar′ may be phenyl, alkylphenyl, methoxyphenyl, aminophenyl, benzoyl, carboxyphenyl, phosphinophenyl or 1,2-(R)(Ar)ethane, where Ar may be phenyl, alkylphenyl, methoxyphenyl, aminophenyl, benzoyl, carboxyphenyl, phosphinophenyl and R may be an alkyl or substituted alkyl.

Alternatively, Y and Y′ may be a Schiff base formed by condensation of an aldehyde (Aid) with an amine (Am). The aldehyde may be an aromatic aldehyde such as for example, pyridine carboxadehyde or a substituted pyridine carboxaldehyde including but not limited to 6-methylpyridinecarboxaldehyde, 5-methylpyridinecarboxaldehyde, 4-methylpyridinecarboxaldehyde or 3-methylpyridinecarboxaldehyde. The amine may be an aromatic amine including but not limited to phenylamine, methoxyphenylamine, ethoxyphenylamine, perfluorophenylamine, sulfonated phenylamine, phosphorylated phenylamine, nitrophenylamine. Those skilled in the art will recognize that a variety of other phenylamines may be also included. The amine may also be an alkylamine, including but not limited to, methylamines, ethylamines, propyl amines, butylamines, pentylamines, hexylamines and cyclic alkylamines such as cyclohexylamine and bicyclohexylamine.

In one embodiment, the role of the bidentate ligand Y-L-Y′ may instead be served by two separate ligands L and L′, which are members of a broad range of ligands known as datively bonded, covalent donor ligands, including but not limited to, organophosphines, carbon monoxide ligands and amine ligands.

When the gas separation agent illustrated in formula (2) is bonded to an olefin during separation of the olefin from an olefin/paraffin stream, the olefin gas separation agent may have a formula comprising:

[(olefin)_{c}M(Xₐ)(X_{b})(Y-L-Y′)]ⁿz[counterion]^{m} (3)

In the embodiment illustrated by formula (3), M is a metal having a vacant coordination site when the gas separation agent is unbound. In other words, the vacant coordination site may be used for associatively binding an olefin to separate the olefin from a paraffin. Xₐ is at least one of a mono-anionic ligand or a donor ligand, X_{b} is at least one of a mono-anionic ligand or a donor ligand and Y-L-Y′ is a bidentate non-anionic ligand having two moieties Y and Y′. In formula (3), a, b and c are integers greater than or equal to zero and n, m and z have a charge balance relationship of n+zm=0.

In one embodiment, c is typically either zero or 1, depending on whether the gas separation agent is in its unbound or bound form, respectively. In some embodiments, c may be greater than 1. In one embodiment, the olefin may be ethylene or propylene.

When Xₐ and X_{b} are both present and are monoanionic ligands bound to Co, Rh or Ir, the charge on the gas separation agent is n=−1 and the product zm is 1. For example, the value of m may be equal to +1 and the value of z may be equal to 1. When Xₐ and X_{b} are both present and are monoanionic ligands bound to Pd or Pt, the charge on the gas separation agent is n=0 and the values of m and z are zero. When Xₐ and X_{b} are both present and are donor ligands bound to Co, Rh or Ir, the charge on the gas separation agent is n=+1 and z=1 and m=−1. When Xₐ and X_{b} are both present and are donor ligands bound to Pd or Pt, the charge on the gas separation agent is n=+2 and product zm=−2 (for example, m=−2 and z=1 or m=−1 and z=2). When Xₐ is a donor ligand bound to Co, Rh or Ir and X_{b} is absent, the charge on the gas separation agent is n=−1 and the values of m and z are +1 and 1, respectively. When Xₐ is a donor ligand bound to Pd or Pt and X_{b} is absent, the charge on the gas separation agent is n=0 and the values of m and z are both 0. When Xₐ is a monoanionic ligand bound to Pd or Pt and X_{b} is absent, the charge on the gas separation agent is n=−1 and the values of m and z are +1 and 1, respectively.

The ligands Xₐ and X_{b} may also encompass a single, chelating-dianionic ligand containing anionic moieties Xₐ and X_{b} that are the same or different from one another, written as Xₐ-L′-X_{b} and depicted by formula (4) as:

[(olefin)_{c}M(Xₐ-L′-X_{b})(Y-L-Y′)]ⁿz[counterion]^{m} (4)

In the embodiment illustrated by formula (4), M is a metal having a vacant coordination site for associatively binding an olefin to separate the olefin from a paraffin, Xₐ-L′-X_{b} is a single, chelating dianionic ligand containing anionic moieties Xₐ and X_{b} that are the same or different from one another and Y-L-Y′ is a bidentate non-anionic ligand having two moieties Y and Y′. In formula (4), a, b and c are integers greater than or equal to zero and n, m and z have a charge balance relationship of n−zm=0 or zm−n=0. In one embodiment, c is typically either zero or 1, depending on whether the gas separation agent is being drawn in its unbound or bound form, respectively. In some embodiments, c may be greater than 1. In one embodiment, the olefin may be ethylene or propylene.

Examples of Xₐ-L′-X_{b} include but are not limited to 1,2- or 1,3-substituted diolate ligands, diamide ligands, disulfide ligands, diphosphinate ligands, diphosphate ligands, or structural hybrids thereof; wherein the back bone is a saturated or unsaturated hydrocarbon. The Xₐ and X_{b} in Xₐ-L′-X_{b} may be nicogenide or chalcogenide anionic groups in an ortho-substitution pattern on an aromatic ring, including but not limited to a catecholate or a catecholate having substituents on the aromatic ring, a 1,2-diamidobenzene or a 1,2-diamidobenzene having substituents on the aromatic ring, a 1,2-bis(arsenate)benzene or a 1,2-bis(arsenate)benzene having substituents on the aromatic ring, a 1,2-bis(phosphinate)benzene or a 1,2-bis(phosphinate)benzene having substituents on the aromatic ring, a 1,2-(disphosphonate)benzene or a 1,2-(diphosphonate)benzene having substituents on the aromatic ring or a 1,2-bis(thiolate)benzene or a 1,2-bis(thiolate)benzene having substituents on the aromatic ring.

Although the above embodiments describe various combinations of the ligands and ligand functional group moieties as described above, the present invention should not be limited to any particular combination of these moeities. For example, one embodiment of the present invention may encompass a structure wherein the moieties Xₐ and Y are contained within one chelating ligand and the moieties X_{b} and Y′ are contained within another chelating moiety as in formula (5):

[(olefin)_{c}M(Xₐ-L₁-Y)(X_{b}-L₂-Y′)]ⁿz[counterion]^{m} (5)

Another embodiment involves a dimeric or polymeric structure capable of binding and separating more than one molecule of olefin per molecule of separating agent as in formula (6):

[{(olefin)_{c}}M₁(Xₐ)(X_{b})(μ-Y-L-Y′)M₂(Xₐ)(X_{b})(μ-Y-L-Y′) . . . Mₚ(Xₐ)(X_{b})]ⁿz[counterion]^{m} (6)

Wherein Xₐ and X_{b} are defined above and μ-Y-L-Y′ is as defined above for Y-L-Y′, wherein μ-Y-L-Y′ is capable of forming a covalent bridge between metal atoms and Xₐ and X_{b} are capable of forming a covalent bridge between metal atoms; and the value of c is greater than or equal to the number of bridged metal atoms P.

In another embodiment, the novel gas separation agent comprises a gas separation agent having a formula comprising:

[ML₁L₂L₃L₄]ⁿz[anion]^{m} (7)

In the embodiment illustrated by formula (7), M is a metal having a vacant coordination site when the gas separation agent is unbound. The vacant coordination site may be used for associatively binding an olefin to separate the olefin from a paraffin. In other words, the metal M does not a priori require a bond to be dissociated before binding an olefin. L₁-L₄ are each a member of a broad range of ligands known as datively bonded, covalent donor ligands including, but not limited to, organophosphines, carbon monoxide ligands and amine ligands. The anion may be any ion for balancing a charge of the gas separation agent. In formula (7), n, m and z have a charge balance relationship of n+zm=0.

The novel gas separation agent of formula (7) above selectively and reversibly binds an olefin. The novel gas separation agent of formula (7) above selectively binds an olefin because the novel gas separation agent tends to only bind with an olefin from an olefin/paraffin feed stream. The novel gas separation agent of formula (7) above reversibly binds an olefin because a reaction of the olefin and the novel gas separation agent may go in either direction.

When the gas separation agent illustrated in formula (7) is bonded to an olefin during separation of the olefin from an olefin/paraffin stream, the olefin gas separation agent may have a formula comprising:

[(olefin)_{c}ML₁L₂L₃L₄]ⁿz[anion]^{m} (8)

In the embodiment illustrated by formula (8), M and L₁-L₄ as described above. In formula (8), c is an integer greater than or equal to one. In one embodiment, c is 1. In some embodiments, c may be greater than 1. In one embodiment, the olefin may be ethylene or propylene.

In one embodiment, the metal Nickel (Ni) may be used according to the formulae described above for the metals Pd and Pt. When using Ni, the gas separation agents will generally have a tetrahedral geometry in the unbound form.

In one embodiment, the present invention uses a reversible insertion of an olefin into a metal hydrogen bond as a separation mechanism. For example, one of Xₐ or X_{b} described above may be a hydride (H⁻) ligand.

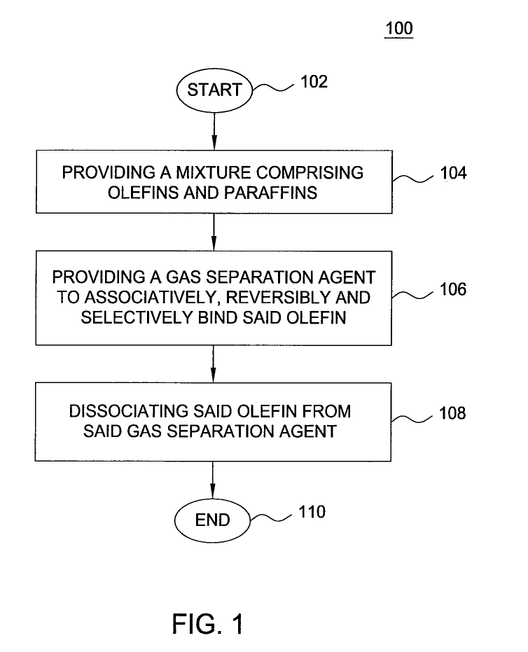

FIG. 1 illustrates a flow diagram of one embodiment of a method 100 for separating olefins from paraffins. The method 100 begins at step 102. At step 104, the method 100 provides a mixture comprising olefins and paraffins.

At step 106, the method 100 provides a gas separation agent to associatively and reversibly bind said olefin. Associatively is defined as not requiring that a ligand leave the gas separation agent prior to olefin binding. Reversibly is defined as allowing the reaction (i.e. the binding of the olefin to the gas separation agent) to go in either direction. That is, the olefin may come “on” and “off” of the gas separation agent. Selectively is defined as tending to bind with a specific species from a stream of multiple species (e.g., the gas separation agent tends to only bind with the olefin from an olefin/paraffin feed stream). For example, any one of the novel gas separation agents described above may be used.

At step 108, the method 100 dissociates the olefin from the gas separation agent via a reversible reaction. The method 100 may be carried out in a variety of different separation processes, using the novel gas separation agents in a solution phase, a liquid phase, a gel phase or a solid phase. For example, the method may be carried out in a solution phase separator, a solid phase separator, a flash pot separator, a counter-current liquid phase separator, a solid phase tubular separator, a pressure swing sorption separator, a temperature swing sorption separator, an electrochemical swing absorption separator, or any combination thereof. Those skilled in the art will recognize that other suitable separation processes may exist and the present invention is not limited to the list of separations processes provided above. The method 100 concludes at step 110.

### EXAMPLES

The following examples describe the invention in further detail. However, the following examples are not intended to limit the scope of the present invention.

### Example 1

A solution of PtCl₂(diMePhen) in deuterated dichloromethane (CD₂Cl₂) was saturated with ethylene (diMePhen=2,9-dimethyl-1,10-phenanthroline). After 16 hours (hr) standing under 1 atm of ethylene, the fraction of Pt bound to ethylene was measured as 75%. The solution was then degassed for several minutes by bubbling argon through it, whereupon the fraction of Pt bound to ethylene was measured immediately as 30%. The solution was then saturated with ethylene once again, and the fraction of Pt bound to ethylene was measured immediately as 46%. This example shows that ethylene binds reversibly to the gas separation agent in a non-coordinating solvent. The criteria of reversibility and associative mechanism are therefore satisfied and PtCl₂(diMePhen) is a candidate for gas separations in solution or in the solid state, based on the mechanism of gas uptake and release.

### Example 2

The gas separation agent PtCl₂(diMePhen) was placed in an NMR tube (7.64 mg, 0.0154 mmol), deoxygenated under vacuum, and dissolved in 1.5 milliliters (mL) CD₂Cl₂. Ethylene was bubbled through the solution for several minutes and the tube was sealed under 1 atm ethylene atmosphere and allowed to stand for 10 hr. The volume of the solution after treatment with ethylene was 1.0 mL.

The relative quantity of unbound gas separation agent was determined by integration of its methyl resonance (6=3.23 ppm, 6H). The quantity of bound gas separation agent was also determined by integration of its methyl resonance (δ=3.48 ppm, 6H). The quantity of bound gas separation agent determined by integration of its ethylene resonance (d=3.61 ppm, 4H) agreed with that calculated from the methyl resonance, to within 1%, and the two values were averaged. The quantity of unbound ethylene was determined by integration of its resonance at δ=5.41 ppm. These three concentrations were substituted into equation (1), above, to give an equilibrium constant value for K_{bind} of 610 M⁻¹.

The measured value of K_{bind} may vary somewhat from one solvent to another or from solution to solid state. Because the measured value of K_{bind} of 610 M⁻¹ is between about 1 and 1000 M⁻¹, the gas separation agent PtCl₂(diMePhen) is expected to be useful for gas separations in solution or in the solid state, based on the thermodynamics of gas uptake and release.

### Example 3

A solution of PdCl₂(diMePhen) in CD₂Cl₂ was saturated with ethylene. After 16 hr standing under 1 atm of ethylene, the fraction of Pt bound to ethylene was measured as 10% and the equilibrium value of K_{bind} was measured as 6 M⁻¹. The criteria of reversibility and thermodynamic range of K_{bind} are therefore satisfied.

### Example 4

A quantity of PtCl(CH₃)(t-BUN═CH-2-pyridine) was mixed with an equal mass of polyethylene glycol polymer (also referred to herein as a polyethylene oxide or PEO) in solution phase. A film was cast and dried to a solid and its gas uptake and release properties were analyzed. The uptake of ethylene under ethylene pressure and the release of ethylene under reduced pressure upon repeated cycling indicated that the olefin bonding to the gas separation agent was reversible. The gas uptake and release at various pressures were compared to those of a comparable film cast from just the PEO alone. From these data, the value of K_{bind} for the binding of ethylene to the gas separation agent alone was estimated as 8E-4 atm⁻¹. Reversibility is observed as per the invention, with an equilibrium binding constant lower than desired within the invention.

While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims and their equivalents.

What is claimed is:

1. A gas separation agent for separating an olefin from a paraffin by reversibly and selectively binding said olefin, said gas separation agent represented by the general formula:
[M(Xₐ)(X_{b})(Y-L-Y′)]ⁿz[counterion]^{m},
wherein M is a metal having a vacant coordination site for associatively binding said olefin to separate said olefin from said paraffin;
wherein Xₐ is at least one of: a mono-anionic ligand or a donor ligand;
wherein X_{b} is at least one of: a mono-anionic ligand or a donor ligand;
wherein Y-L-Y′ is a bidentate non-anionic ligand having two moieties Y and Y′;
wherein counterion is an ion for balancing a charge of the gas separation agent;
wherein a and b are integers greater than or equal to zero; and
wherein n, m and z have a charge balance relationship of n+zm=0.

2. The gas separation agent of claim 1, wherein M is a metal selected from a group consisting of: Co, Rh, Ir, Pd, Pt and Ni.

3. The gas separation agent of claim 1, wherein said gas separation agent has an electronic configuration of less than 18 electrons around the metal M when unbound to said olefin.

4. The gas separation agent of claim 1, wherein said gas separation agent comprises a square planar or tetrahedral geometry when unbound to said olefin.

5. The gas separation agent of claim 1, wherein said gas separation agent comprises at least one of: a trigonal bipyramidal geometry or a square pyramidal geometry when associatively bound to said olefin.

6. The gas separation agent of claim 1, wherein when said gas separation agent is associatively bound to said olefin and when Xₐ comprises a monoanionic ligand, X_{b} comprises a monoanionic ligand and M comprises Co, Rh or Ir, n is −1, m is +1 and z=1.

7. The gas separation agent of claim 1, wherein when said gas separation agent is associatively bound to said olefin and when Xₐ comprises a monoanionic ligand, X_{b} comprises a monoanionic ligand and M comprises Pd or Pt, n is 0, m is 0 and z=0.

8. The gas separation agent of claim 1, wherein when said gas separation agent is associatively bound to said olefin and when Xₐ comprises a donor ligand, X_{b} comprises a donor ligand and M comprises Co, Rh or Ir, n is +1, m is −1 and z=1.

9. The gas separation agent of claim 1, wherein when said gas separation agent is associatively bound to said olefin and when Xₐ comprises a donor ligand, X_{b} comprises a donor ligand and M comprises Pd or Pt, n is +2, m is −2 or −1 and z=1 or 2.

10. The gas separation agent of claim 1, wherein said monoanionic ligand comprises a halide.

11. The gas separation agent of claim 10, wherein Xₐ comprises said halide and X_{b} comprises at least one of: an alkyl compound or an aromatic moiety.

12. The gas separation agent of claim 1, wherein said donor ligand comprises at least one of: acetonitrile, dimethylsulfoxide, pyridine, dimethylformamide, methyl amine, ethyl amine, propyl amine, butyl amine, tert-butylamine, methylpyrrolidinone or water.

13. The gas separation agent of claim 1, wherein each one of said two moieties Y and Y′ form a dative donor bond to said metal.

14. The gas separation agent of claim 1, wherein Y and Y′ are identical moieties.

15. The gas separation agent of claim 1, wherein Y and Y′ are different moieties.

16. The gas separation agent of claim 1, wherein Y-L-Y′ comprises a 1,2-disubstituted organic ligand having two substituents, wherein each one of said two substituents are at least one of: a nicogenide or a chalcogenide.

17. A gas separation agent for separating an olefin from a paraffin by reversibly and selectively binding said olefin, said gas separation agent represented by the general formula:
[ML₁L₂L₃L₄]ⁿz[anion]^{m},
wherein M is a metal having a vacant coordination site for associatively binding said olefin to separate said olefin from said paraffin;
wherein each one of L₁-L₄ are a datively bonded, covalent donor ligand;
wherein anion is an ion for balancing a charge of the gas separation agent; and
wherein n, m and z have a charge balance relationship of n+zm=0.

18. The gas separation agent of claim 17, wherein M is a metal selected from a group consisting of: Ru, Os and Fe.

19. The gas separation agent of claim 17, wherein said datively bonded, covalent donor ligand comprises at least one of: organophosphines, carbon monoxide ligands or amine ligands.

20. A method for separating olefins from paraffins, comprising:
providing a mixture comprising olefins and paraffins;
providing a gas separation agent to associatively, reversibly and selectively bind said olefin; and
dissociating said olefin from said gas separation agent.

21. The method of claim 20, wherein said gas separation agent is represented by the general formula:
[M(Xₐ)(X_{b})(Y-L-Y′)]ⁿz[counterion]^{m},
wherein M is a metal having a vacant coordination site for associatively binding said olefin to separate said olefin from said paraffin;
wherein Xₐ is at least one of: a mono-anionic ligand or a donor ligand;
wherein X_{b} is at least one of: a mono-anionic ligand or a donor ligand;
wherein Y-L-Y′ is a bidentate non-anionic ligand having two moieties Y and Y′;
wherein counterion is an ion for balancing a charge of the gas separation agent;
wherein a and b are integers greater than or equal to zero; and
wherein n, m and z have a charge balance relationship of n+zm=0.

22. The method of claim 20, wherein said gas separation agent is represented by the general formula:
[ML₁L₂L₃L₄]ⁿz[anion]^{m},
wherein M is a metal having a vacant coordination site for associatively binding said olefin to separate said olefin from said paraffin;
wherein each one of L₁-L₄ are a datively bonded, covalent donor ligand;
wherein anion is an ion for balancing a charge of the gas separation agent; and
wherein n, m and z have a charge balance relationship of n+zm=0.

23. The method of claim 20, wherein said gas separation agent is selected based upon a tuning algorithm.

24. The method of claim 23, wherein said tuning algorithm comprises one or more parameters comprising at least one of: an equilibrium constant or a kinetic constant.

25. The method of claim 20, wherein said method is carried out in at least one of: a solution phase separator, a solid phase separator, a flash pot separator, a counter-current liquid phase separator, a solid phase tubular separator, a pressure swing absorption (PSA) separator, a temperature swing absorption (TSA) separator, a electrochemical swing absorption separator or any combination thereof.

26. The method of claim 20, wherein said gas separation agent is in at least one of: a solution phase, a liquid phase, a gel phase or a solid phase.

27. The method of claim 20, wherein no acetylene polymers are formed.

28. The method of claim 20, wherein said olefin comprises an unsaturated hydrocarbon.

29. The method of claim 28, wherein said unsaturated hydrocarbon comprises an alkene.

30. The method of claim 20, wherein said paraffin comprises an alkane.
